(19) United States
(12) Patent Application Publication
(10) Pub. No.: US 2016/0303003 A1
(43) Pub. Date: Oct. 20, 2016

(54) SAND REMOVAL PROCESS

(71) Applicant: ATTICUS INDUSTRIES INC, Burlington, WY (US)

(72) Inventors: Alan Troy HALE, Thousand Oks, CA (US); Lane Kristine HALE, Thousand Oaks, CA (US); Jakob BREDSGUARD, Lake Forest, CA (US); Dalin WINTERS, Burlington, WY (US)

(73) Assignee: ATTICUS INDUSTRIES INC, Burlington, WY (US)

(21) Appl. No.: 15/130,759

(22) Filed: Apr. 15, 2016

Related U.S. Application Data
(60) Provisional application No. 62/148,347, filed on Apr. 16, 2015.

(51) Int. Cl. A61K 8/04; A45D 19/00; B08B 7/04; B01J 20/24; B01J 20/04; B01J 20/10; A61Q 19/10; A61Q 5/02; A61K 8/73; A61K 8/19; A61K 8/25; A61K 8/97; A61K 8/67; C09K 3/30; B05B 11/00

(52) U.S. Cl. CPC A61K 8/046; B05B 11/3047; A45D 19/00; B08B 7/04; B01J 20/24; B01J 20/043; B01J 20/10; A61Q 19/10; A61Q 5/02; A61K 8/732; A61K 8/19; A61K 8/25; A61K 8/97; A61K 8/678; C09K 3/30

(57) ABSTRACT

Processes and devices are disclosed for removing sand from a surface, such as skin, using a spray device containing a formula comprising moisture-absorbing powder, propellant, and solvent. The formula is sprayed on a sandy surface using the spray device, and the sandy surface is then wiped to remove sand therefrom.

### RELATED APPLICATION

This application claims priority to U.S. Provisional Application No. 62/148,347, filed on Apr. 16, 2015, entitled FORMULA SPRAY DEVICES AND METHODS, the disclosure of which is hereby incorporated by reference in its entirety.

### BACKGROUND

1. Field

The present disclosure generally relates to sand removal processes.

2. Description of Related Art

Sand can adhere to skin or other surfaces, particularly in the presence of moisture. Removing sand from skin or other surface can be difficult, messy, and/or uncomfortable.

### SUMMARY

In some implementations, the present disclosure relates to a method of removing sand from a surface. The method involves providing a spray device containing a formula comprising moisture-absorbing powder, propellant, and solvent. The method further involves spraying the formula on a sandy surface using the spray device and wiping the sandy surface to remove sand therefrom. The method may further involve waiting a predetermined period of time after said spraying before performing said wiping.

In certain embodiments, the moisture-absorbing powder comprises tapioca starch. The moisture-absorbing powder may further comprise baking soda. In certain embodiments, the moisture-absorbing powder comprises talcum powder. In certain embodiments, the moisture-absorbing powder comprises baking soda. In certain embodiments, the moisture-absorbing powder comprises corn starch. In certain embodiments, the moisture-absorbing powder comprises arrowroot starch.

In certain embodiments, the formula further comprises aloe. In certain embodiments, the formula further comprises lubricant. For example, the lubricant may comprise diisopropyl adipate. In certain embodiments, the formula further comprises acrylate copolymer. In certain embodiments, the formula further comprises caprylyl glycol. In certain embodiments, the formula further comprises silicone. For example, the silicone may comprise dimethicone. In certain embodiments, the formula further comprises aloe. In certain embodiments, the formula further comprises vitamin E.

In some implementations, the present disclosure relates to a sand removal device comprising a nozzle, an actuator, and a bottle containing a formula for removing sand from surfaces. The formula comprises moisture-absorbing powder, propellant, and solvent. In certain embodiments, the formula further comprises lubricant. In certain embodiments, the formula further comprises silicone.

### BRIEF DESCRIPTION OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates a spray bottle/can or device according to one or more embodiments disclosed herein.

FIG. 2 illustrates a process for removing sand from a surface according to one or more embodiments disclosed herein.

FIG. 3 illustrates a process for pre-treating a surface with a spray formula according to one or more embodiments.

### DETAILED DESCRIPTION

While certain embodiments are described, these embodiments are presented by way of example only, and are not intended to limit the scope of protection. Indeed, the novel methods and systems described herein may be embodied in a variety of other forms. Furthermore, various omissions, substitutions and changes in the form of the methods and systems described herein may be made without departing from the scope of protection.

The headings provided herein are for convenience only and do not necessarily affect the scope or meaning of the claimed invention. Disclosed herein are example configurations and embodiments relating to formula spray products generally, as well as methods for using formula spray devices.

### Spray Bottle

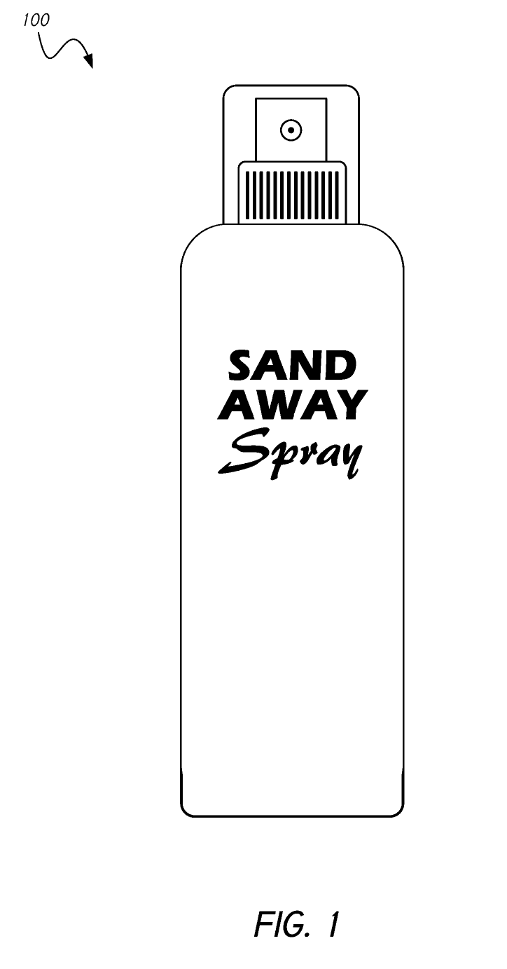

FIG. 1 illustrates a spray bottle/can or device 100 in accordance with one or more embodiments disclosed herein. The bottle 100 may be an aerosol spray bottle or other type of spray bottle configured to dispense a spray formula according to embodiments disclosed herein. The spray bottle 100 may be configured to expel formula in a substantially-continuous, focused spray. The spray bottle 100 may include a valve, nozzle, and/or actuator configured to allow for spraying a various angles, and may be enabled for substantially upside down spraying.

### Spray Formula

Certain embodiments disclosed herein relate at least in part to formula spray devices and/or methods. For example, certain embodiments provide for spray formula comprising one or more of the following ingredients: moisture-absorbing powder or substance, propellant, and/or solvent.

Certain embodiments comprise a moisture-absorbing powder or other substance, which may serve to absorb moisture, cling to particles/debris, or for other purposes. For example, the formula may comprise tapioca starch, baking soda, or talcum powder (i.e., “talc” powder), which may have a chemical composition of, for example, H₂Mg₃(SiO₃)₄ or Mg₃Si₄O₁₀(OH)₂. Additionally, or alternatively, other moisture-absorbing substances may be included in the formula, such as corn starch, which may comprise a starch derived from the corn (i.e., maize) grain and may be derived from the endosperm of the corn kernel; or arrowroot powder or flour. Arrowroot, as used herein, may refer to a starch obtained from the rhizomes (rootstock) of one or more of various plants, such as maranta arundinacea, zamia pumila, tapioca from cassava (manihot esculenta), pueraria lobate (“Japanese arrowroot”), and/or other substances. With respect to sand-removal solutions, the moisture-absorbing substance may absorbs any moisture or oil in the sand or on the skin (or any other object), which may serve to at least partially release the sand. Without a certain amount of moisture or an oily surface, the sand may no longer stick to the same degree to the skin/surface, and may therefore be relatively easily removed.

Certain embodiments comprise one or more propellants, such as Isobutene, hydrofluorocarbon (e.g., Hydrofluorocarbon 152A), or other type of organofluorine compound. The propellant may comprise a pressurized gas substantially in equilibrium with its liquid (e.g., at its saturated vapour pressure). When used in a spray can application, as disclosed herein, liquid evaporation in connection with expulsion of gas may help maintaining relatively even pressure during device use and over time.

Certain embodiments comprise one or more chemical solvents. For example, the formula may comprise one or more alcohol compounds, such as ethanol, or other relatively low-weight alcohol, which may serve to promote even spreading of the spray formula, dissolve surface oils, and/or improve skin feel. In certain embodiments, alcohol having relatively-low boiling point is used to promote evaporation to allow the formula to dry relatively quickly on skin or other surface. Certain embodiments may comprise one or more of the following compounds: Alcohol Denat., SD Alcohol 3-A, SD Alcohol 30, SD Alcohol 39, SD Alcohol 39-B, SD Alcohol 39-C, SD Alcohol 40, SD Alcohol 40-B, SD Alcohol 40-C, Isododecane, or the like.

Certain embodiments may include one or more additional ingredients, such as: kaolinite (e.g., kaolin clay), bentonite clay, sodium bentonite, calcium bentonite, potassium bentonite, or the like, which may advantageously provide moisture absorption and/or impurity removal functionality; calendula, chamomile, aloe, lavender and/or other herb or substance, which may be in power or other form, and may provide anti-inflammatory, antimicrobial, astringent, antifungal, and/or other properties.

The spray formula may further comprise one or more of the following ingredients: Diisopropyl Adipate, or other lubricant; VA/Butyl Maleate/Isobornyl Acrylate Copolymer, or other copolymer containing the monomers acrylic acid or methacrylic acid or salts or esters thereof, or other moisture-resistant polymer; Caprylyl Glycol, or other skin-conditioning agent; Dimethicone, or polydimethylsiloxane (PDMS), or other silicone, or silicon-based organic polymer, or silicone oil (e.g., polymerized siloxane); Ascorbyl Palmitate, or other Vitamin C derivative, Tocopheryl Acetate, or other Vitamin E derivative; Mineral Oil; Panthenol, or other provitamin; Water, Aloe Barnadensis Leaf Extract, or other aloe-based supplement; Fragrance; and/or other ingredient(s).

### Sand Removal Processes

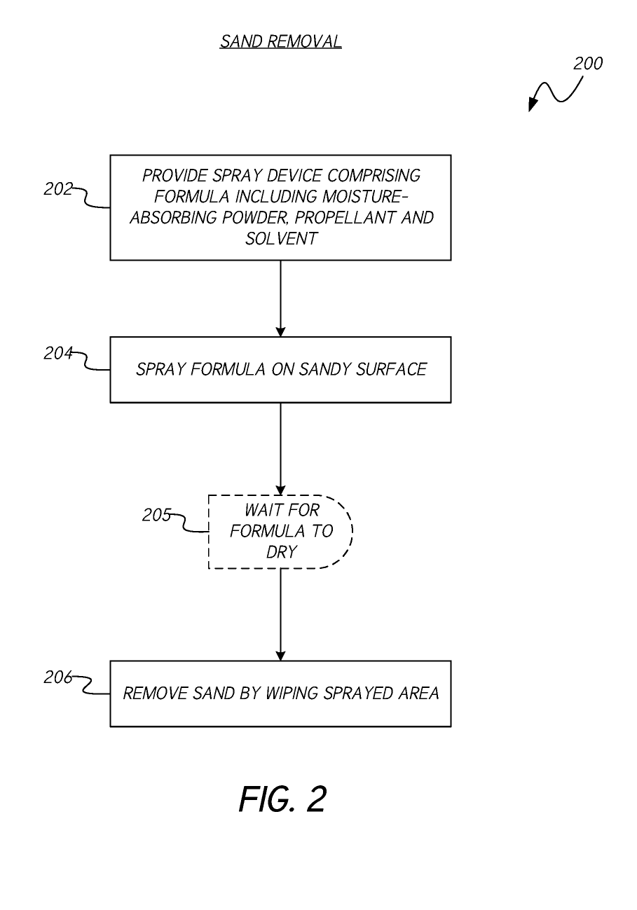

FIG. 2 illustrates a process 200 for removing sand from a surface, such as a skin surface. At block 202, the process 200 involves providing a spray device comprising a spray formula including one or more of a moisture-absorbing powder or other substance, propellant, and/or solvent, as described above. In certain embodiments, the spray device comprises an aerosol spray can, which may have any suitable or desirable volume, size, or attributes.

At block 204, the process 200 involves spraying the formula of the spray device onto a sandy surface, such as onto a skin surface of a body member of the user or other individual. For example, or legs, feet, arms, or other parts of an individual's body may be at least partially covered with sand, or have an amount of sand disposed thereon. Such sand may be at least partially adhered to the skin surface, or other surface, when water or other liquid is mixed with the sand to allow the sand to cling or adhere to the surface. The formula may be sprayed substantially evenly over at least a portion of the sandy surface, and may be allowed to at least partially dry. In certain embodiments, certain ingredients or components of the formula may cling or bond to particles, such as sand or other debris as a result of said spraying.

At block 206, the process 200 involves removing at least a portion of the sand on the sandy surface by applying frictional force to said sand, such as in a wiping motion or the like. Such wiping may be performed with, for example, a hand, towel, or other tool or device. In certain embodiments, said wiping may result in at least a portion of the sand of the sandy surface being substantially removed from the area wiped, and may leave the area substantially clean. In certain embodiments, a formula residue may be left on the surface in some amount or area.

In some embodiments, the process 200 may further involve waiting for a period of time for the formula to dry prior to wiping the sprayed surface/area, as shown at block 205, which may improve the sand-removing effect of the formula 200. For example, the period of time may be a predetermined period of time. In certain embodiments, waiting, as shown at block 205, may involve waiting approximately 2 seconds, 5 seconds, 10 seconds, or other period of time.

### Hair Cleaning Processes

Certain embodiments disclosed herein provide processes for cleaning hair or other substances using spray formula as described herein. For example, a process for cleaning hair may involve spraying a formula including one or more of a moisture-absorbing substance, propellant, and/or solvent onto hair or other substance, wherein said formula is configured to at least partially absorb certain oils and/or other substances, and/or provide other benefits to the hair or other substance.

The spray application of the hair-cleaning formula may advantageously allow for application of the formula to the hair other substance from various angles, and in a substantially even spray radius and/or volume. The hair cleaning processes described herein may therefore provide for use of the spray can including formula as described herein as a dry shampoo application, wherein water is not necessary to provide cleaning of the hair other substance.

### Skin Pre-Treatment Processes

Although certain embodiments disclosed herein are described in the context of spraying sand on a surface or substance in order to provide cleaning functionality, wherein particles and/or debris or other substances are effectively absorbed and/or removed from the target surface or substance, spray formula applications as described herein may advantageously be used, in certain embodiments, as a prophylactic solution to protect against particles, oils, debris, or other contaminant or contamination of a surface or substance.

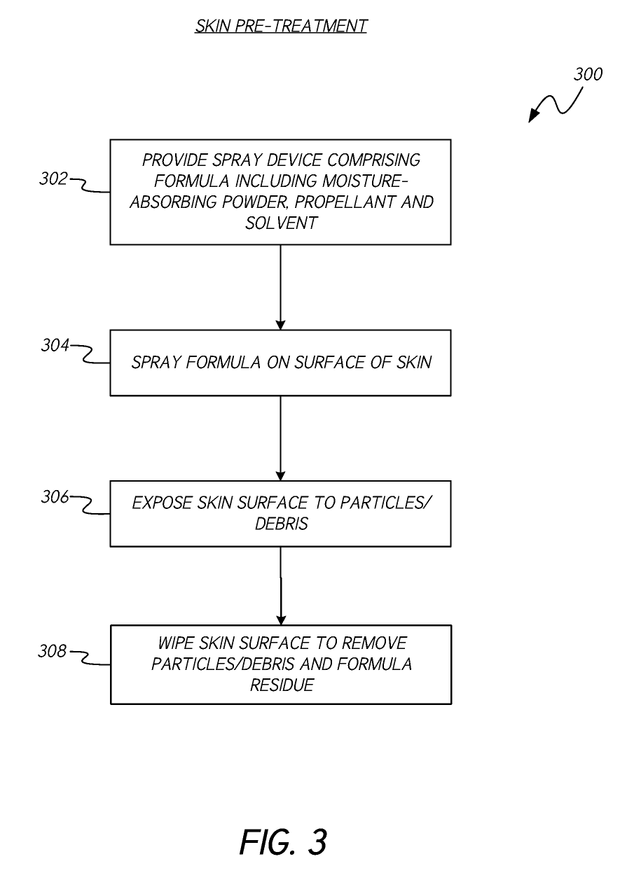

For example, FIG. 3 illustrates a process 300 for pre-treating a surface or substance with the spray formula as described herein. The process 300 involves, at block 302, providing a spray device comprising formula including one or more of a moisture-absorbing substance (e.g., powder), propellant, and/or solvent (e.g., alcohol), as described in greater detail above. At block therefore, the process 300 involves spraying formula using the spray device on a surface of skin, or other surface or substance, to at least partially protect or prevent from the adhesion or contamination of various particles, substances, debris, or other material or substance on the sprayed surface or substance.

At block 306, the process 300 involves exposing the skin surface or other surface or substance to various particles, debris, contaminant or other substance, which it may be desirable to protect against. Examples of embodiments or contexts in which surface or skin pretreatment according to the process 300 may be desirable may include, for example, protection of skin from sand, protection of skin pieces of hair, which may cause irritation when encountered in connection with haircutting, handling of pets, or other activity. Additional examples may include, for example, protection from adhesion or irritation of fiberglass insulation particles, which may be encountered during installation of fiberglass insulation, or other applications.

At block 308, the process 300 involves wiping the skin surface or other surface or substance particles, debris, or other substance or contaminant from the surface of skin or other substance. Said wiping may further serve to at least partially remove residue associated with the spray formula.

### Additional Embodiments

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Description using the singular or plural number may also include the plural or singular number respectively. The word “or” in reference to a list of two or more items, that word covers all of the following interpretations of the word: any of the items in the list, all of the items in the list, and any combination of the items in the list.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel, or may be performed at different times.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

The teachings of the invention provided herein can be applied to other systems, not necessarily the system described above. The elements and acts of the various embodiments described above can be combined to provide further embodiments.

While some embodiments of the inventions have been described, these embodiments have been presented by way of example only, and are not intended to limit the scope of the disclosure. Indeed, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

What is claimed is:

1. A method of removing sand from a surface, the method comprising:
providing a spray device containing a formula comprising:
moisture-absorbing powder;
propellant; and
solvent;
spraying the formula on a sandy surface using the spray device; and
wiping the sandy surface to remove sand therefrom.

2. The method of claim 1, further comprising waiting a predetermined period of time after said spraying before performing said wiping.

3. The method of claim 1, wherein the moisture-absorbing powder comprises tapioca starch.

4. The method of claim 3, wherein the moisture-absorbing powder further comprises baking soda.

5. The method of claim 1, wherein the moisture-absorbing powder comprises talcum powder.

6. The method of claim 1, wherein the moisture-absorbing powder comprises baking soda.

7. The method of claim 1, wherein the moisture-absorbing powder comprises corn starch.

8. The method of claim 1, wherein the moisture-absorbing powder comprises arrowroot starch.

9. The method of claim 1, wherein the formula further comprises aloe.

10. The method of claim 1, wherein the formula further comprises lubricant.

11. The method of claim 10, wherein the lubricant comprises diisopropyl adipate.

12. The method of claim 1, wherein the formula further comprises acrylate copolymer.

13. The method of claim 1, wherein the formula further comprises caprylyl glycol.

14. The method of claim 1, wherein the formula further comprises silicone.

15. The method of claim 14, wherein the silicone comprises dimethicone.

16. The method of claim 1, wherein the formula further comprises aloe.

17. The method of claim 1, wherein the formula further comprises vitamin E.

18. A sand removal device comprising:
a nozzle;
an actuator; and
a bottle containing a formula for removing sand from surfaces, the formula comprising:
moisture-absorbing powder;
propellant; and
solvent.

19. The sand removal device of claim 18, wherein the formula further comprises lubricant.

20. The sand removal device of claim 18, wherein the formula further comprises silicone.
